(19) United States
(12) Patent Application Publication
(10) Pub. No.: US 2022/0218384 A1
(43) Pub. Date: Jul. 14, 2022

(54) VIBRATION TYPE REMOVAL APPARATUS

(71) Applicant: MICRON MACHINERY CO., LTD., YAMAGATA-SHI, YAMAGATA (JP)

(72) Inventors: Satoshi KOBAYASHI, YAMAGATA-SHI, YAMAGATA (JP); Yoshihiro MINAGAWA, YAMAGATA-SHI, YAMAGATA (JP); Hiroki SUZUKI, YAMAGATA-SHI, YAMAGATA (JP); Makoto SAKAKIBARA, YAMAGATA-SHI, YAMAGATA (JP)

(21) Appl. No.: 17/613,280

(22) PCT Filed: Apr. 22, 2020
(86) PCT No.: PCT/JP2020/017411

§ 371 Date: Nov. 22, 2021

(30) Foreign Application Priority Data
Mar. 10, 2020 (JP) 2020-040674

(51) Int. Cl. A61B 17/32

(52) U.S. Cl. CPC A61B 17/320068; A61B 17/320708

(57) ABSTRACT

A vibration type removal apparatus 2 includes a housing 10, a holding member 11, a tool 12, a control device 20, a vibration device 21, and a cooling pump 22. The control device 20 drives the cooling pump 22, and cooling water supplied from the cooling pump 22 passes through a flow path 25 in the housing 10, and is sprayed toward the tool 12 from a distal end of the flow path 25. In this state, the tool 12 is inserted between a hard tissue HT and a soft tissue ST, the vibration device 21 is operated, and the tool 12 is vibrated. Due to the vibration of the tool 12, a surface of the hard tissue HT which adheres to the soft tissue ST is cut, and the hard tissue HT is peeled and removed from the soft tissue ST.

### TECHNICAL FIELD

The present invention relates to a vibration type removal apparatus that peels and removes a hard tissue from a soft tissue using a tool attached to its distal end portion.

### BACKGROUND ART

A vibration type cutting apparatus that comprises a vibration device and cuts an object to be removed by vibrating a tool attached to a distal end portion of the vibration device has been known (e.g., Patent Literature 1).

In the vibration type cutting apparatus described in Patent Literature 1 and a general rotation type cutting apparatus, vibration of a tool by a vibration device is controlled to burst oscillation in which stop and vibration are repeated, to improve operability.

In the vibration type cutting apparatus described in Patent Literature 1, when surgery for removing a hard tissue adhering to a soft tissue is performed, the hard tissue is cut using the tool from the side of a surface opposite to its surface adhering to the soft tissue, and the hard tissue is cut until the thickness thereof is a small thickness, e.g., a thickness of approximately one sheet. The thinned hard tissue is carefully manually removed by a handpiece such as a curette.

### CITATION LIST

### Patent Literature

Patent Literature 1: Japanese Patent Laid-Open No. 2019-088452

### SUMMARY OF INVENTION

### Technical Problem

However, when a hard tissue is cut until it becomes thin, an unintended deviation of a distal end of a tool easily leads to a damage to a soft tissue because a force is applied to the tool in a direction in which the soft tissue exists. Particularly when a rotary tool is used, frictional heat generated on a contact surface with a thin hard tissue produces the possibility of damaging a soft tissue closest to the hard tissue. Thus, surgery (cutting work) needs to be carefully performed, so that a surgery time period is lengthened.

The present invention has been made in view of the foregoing points, and is directed to providing a vibration type removal apparatus capable of efficiently peeling and removing a hard tissue from a soft tissue.

### Solution to Problem

[1] A vibration type removal apparatus according to the present invention is a vibration type removal apparatus that peels and removes a hard tissue from a soft tissue, the vibration type removal apparatus comprising a housing, a vibration device arranged in the housing, a tool provided in a distal end portion of the vibration device, protruding from the housing, and insertable between the hard tissue and the soft tissue, a control device that controls driving of the vibration device to vibrate the tool in an axial direction of the vibration device, and a cooling device that cools the tool, in which the control device controls the vibration of the tool by the vibration device to burst oscillation in which stop and vibration are repeated.

According to the present invention, the tool provided in the distal end portion of the vibration device and protruding from the housing is insertable between the hard tissue and the soft tissue. Thus, when the tool is inserted between the hard tissue as an object to be removed and the soft tissue and is vibrated with a force applied in a direction away from the soft tissue, the hard tissue can be peeled and removed from the soft tissue by cutting a surface, which adheres to the soft tissue, of the hard tissue. As a result, the hard tissue can be removed in a shorter time period and more stably than when the hard tissue is cut until it becomes thin.

The tool is cooled by the cooling device, and the vibration of the tool is further controlled to burst oscillation in which stop and vibration are repeated. Thus, the tool can be more prevented from generating heat so that heat to be applied to the soft tissue and a peripheral tissue can be more suppressed than when the tool is always vibrated.

[2] A first surface, which opposes the soft tissue, of the tool preferably has a plurality of recesses or a groove extending toward a distal end side from a proximal end side of the tool formed thereon.

This configuration makes it possible to more efficiently cool the tool than when the first surface, which opposes the soft tissue, of the tool is a flat surface having no unevenness.

[3] A second surface, which opposes the hard tissue, of the tool is preferably formed in a circular arc shape, and also a planar shape or a concave shape.

This configuration makes it easier to insert the tool between the hard tissue and the soft tissue than when the second surface, which opposes the hard tissue, of the tool has a projection shape.

[4] The cooling device is preferably composed of a cooling liquid supply device that supplies a cooling liquid, and the housing is preferably provided with a flow path that causes the cooling liquid supplied from the cooling liquid supply device to flow toward a distal end side of the tool. A distal end of the flow path is preferably provided to spray the cooling liquid toward a distal end portion of the tool.

This configuration makes it possible to cool the tool with cooling liquid supplied from the cooling liquid supply device and flowing through the flow path, and thus makes it possible to more efficiently cool the tool.

### BRIEF DESCRIPTION OF DRAWINGS

FIG. 1 is an explanatory view related to a configuration of a vibration type removal apparatus according to an embodiment of the present invention.

FIG. 2A is a perspective view illustrating a tool.

FIG. 2B is a perspective view illustrating the tool.

FIG. 3A is a schematic view illustrating the tool, a hard tissue, and a soft tissue.

FIG. 3B is a schematic view illustrating a state where the tool is inserted between the hard tissue and the soft tissue.

FIG. 3C is a schematic view illustrating a state where a hard tissue is peeled and removed using the tool.

FIG. 4 is a perspective view illustrating a tool having a plurality of recesses formed therein.

### DESCRIPTION OF EMBODIMENT

An embodiment of the present invention will be described below with reference to the drawings.

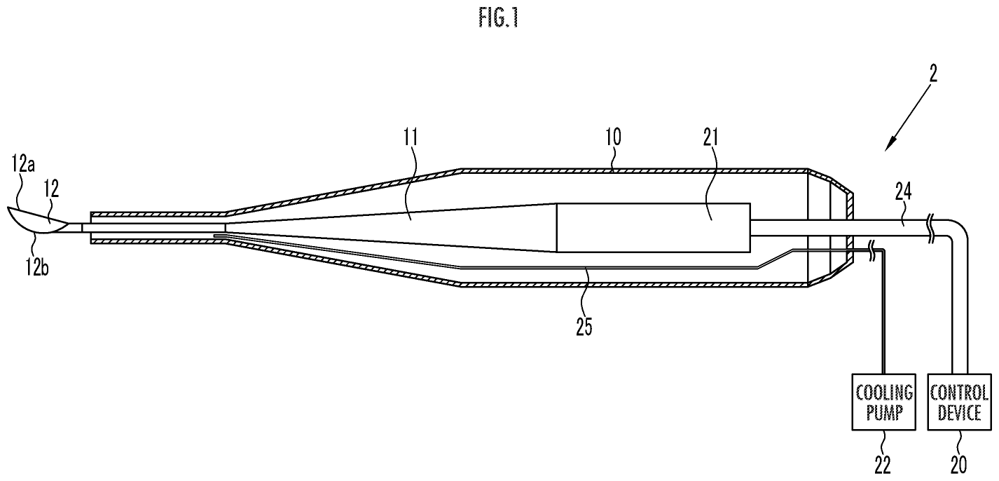

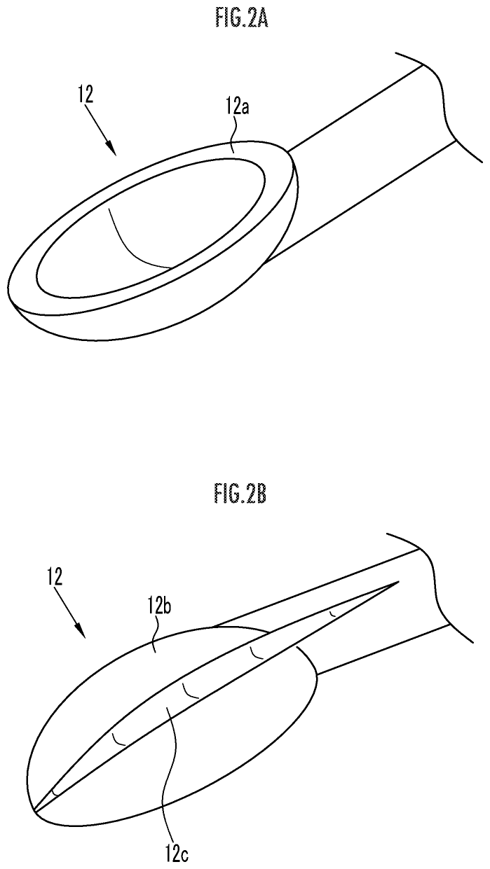

As illustrated in FIG. 1, a vibration type removal apparatus 2 according to an embodiment of the present invention comprises a housing 10 having a substantially cylindrical shape, a holding member 11, a tool 12, a control device 20, a vibration device 21, and a cooling pump 22, which constitute a handpiece.

The housing 10 is designed to a size small enough for a normal person to hold it with one hand. Although a handpiece as a high-frequency vibration type cutting apparatus is constituted by components such as the housing 10 and the holding member 11 at least a part of which is arranged in an inner space of the housing 10, the respective types and specifications of the components may be appropriately selected from the viewpoint of making the handpiece lightweight for simplicity of handling.

The holding member 11 has its rear end portion attached to the vibration device 21, and is supported to be movable in its axial direction on the housing 10 via a support section (not illustrate) fixed to an inner sidewall of the housing 10.

The holding member 11 has a function as a horn for increasing an amplitude. The tool 12 is detachably attached to a distal end portion of the holding member 11.

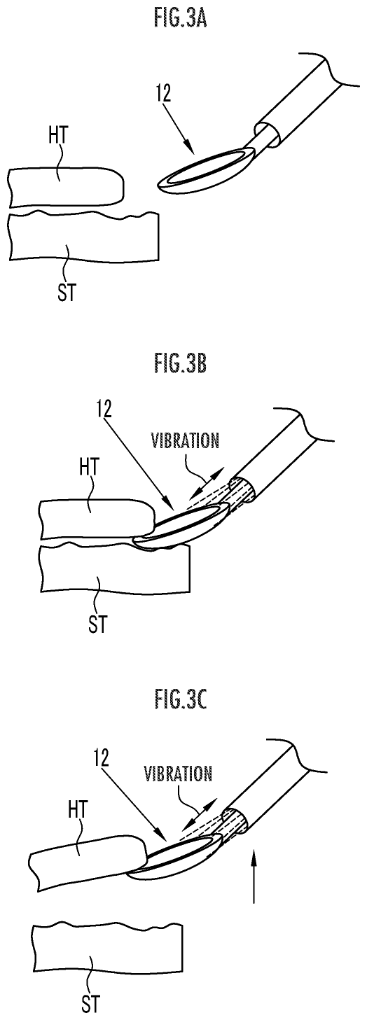

The tool 12 may be easily insertable between a hard tissue HT and a soft tissue ST (see FIG. 3B), and examples of the type of the tool 12 include a curette, a chisel, a scalpel, a file, a long type, and a short type. A blade having a linear shape or a circular arc shape at its distal end or a tool having any shape such as a substantially columnar shape, a spoon shape, or a bent or curved rod shape is adopted as the tool 12.

In the present embodiment, the tool 12 is composed of a curette, a distal end portion of the tool 12 has a hemispherical shape, a first surface 12a, which is obtained by cutting a sphere in half, of the tool 12 is formed in a circular arc shape and a concave shape, and a groove 12c extending toward the distal end portion from a proximal end portion on the holding member 11 side of the tool 12 is formed on a second surface 12b as a hemispherical portion of the tool 12.

The vibration device 21 is attached to an attachment section (not illustrated) in the housing 10 and is composed of a piezoelectric element arranged in the inner space of the housing 10 so that the holding member 11 is vibrated or driven to reciprocate in the axial direction.

The vibration device 21 and the holding member 11 are arranged such that their respective axes are common and arranged away from each other in their respective axial directions. Accordingly, spaces respectively occupied by the vibration device 21 and the holding member 11 in the inner space of the housing 10, and thus the housing 10 can be made more compact in a direction perpendicular to the axes than when the axes are arranged parallel to and away from each other or arranged nonparallel to each other. As a result, a vibration type removal apparatus can be configured as a handpiece ease of handling and operability of which are improved.

A force of the vibration device 21 is directly transmitted to the holding member 11 without a transmission mechanism being used. Accordingly, a lubricant such as grease to generally be used for the transmission mechanism is not required. Therefore, when the vibration type removal apparatus 2 as medical equipment is subjected to sterilization treatment with high-pressure steam, a situation where contamination of the medical equipment derived from the existence of the lubricant occurs is avoided.

The control device 20 is connected to the vibration device 21 via a cable 24 attached to a rear end portion of the housing 10, to control supply of power to the vibration device 21.

The control device 20 controls an operation of the vibration device 21. The control device 20 is composed of a microcomputer or a processor. The control device 20, together with a substrate on which it is mounted, may be arranged in the inner space of the housing 10.

The control device 20 performs control such that a vibration frequency f2 in an axial direction of the tool 12 via the holding member 11 by the vibration device 21 falls within a range of 20 to 60 [kHz]. The vibration frequency f2 is more preferably controlled to 25 to 45 [kHz].

Further, the control device 20 controls the vibration of the tool 12 by the vibration device 21 to burst oscillation in which stop and vibration are repeated, and an overall burst frequency f1 of the tool 12 in which a burst period during which the tool 12 is vibrated and a stop period during which the tool 12 is stopped are combined into one cycle is controlled depending on the cooling efficiency of the tool 12.

With the vibration type removal apparatus 2 having the above-described configuration, the holding member 11 is driven to reciprocate in the axial direction, whereby an object is cut using the tool 12 provided in the distal end portion of the holding member 11.

The control device 20 controls the vibration of the tool 12 by the vibration device 21 to burst oscillation in which vibration and stop are repeated.

Specifically, the control device 20 controls the burst frequency f1 of the tool 12 to a range of 1 to 300 [Hz] as a low frequency at which an operator can recognize a cycle of a burst period and a stop period, and controls a duty ratio dl obtained by dividing a pulse width t1 of the burst frequency by a pulse period T1 to a range of 5 to 50 [%].

The cooling pump 22 sends out (supplies) cooling water sent from a cooling water supply source (not illustrated), and the driving thereof is controlled by the control device 20.

A flow path 25 that causes cooling water supplied from the cooling pump 22 to flow is attached to the rear end portion of the housing 10. The flow path 25 extends to a distal end portion from the rear end portion of the housing 10.

### [Peeling and Removal]

A case where the hard tissue HT as an object to be removed is peeled and removed using the vibration type removal apparatus 2 will be described.

As illustrated in FIG. 3A, the operator who performs peeling and removal (surgery) first attaches the tool 12 to the distal end portion of the holding member 11.

Then, the operator causes the control device 20 to drive the cooling pump 22, as illustrated in FIG. 3B. As a result, cooling water supplied from the cooling pump 22 passes through the flow path 25 in the housing 10, and is sprayed toward the tool 12 from a distal end of the flow path 25. In this state, the operator inserts the tool 12 between the hard tissue HT and the soft tissue ST. In FIG. 3B and FIG. 3C, cooling water is indicated by a dotted line for simplification.

As illustrated in FIG. 3C, the operator causes the control device 20 to operate the vibration device 21, to drive the holding member 11 to reciprocate in the axial direction. As a result, the tool 12 provided in the distal end portion of the holding member 11 is driven to reciprocate (vibrated) in the axial direction. At this time, the operator applies a force in a direction away from the soft tissue ST (in an upward direction in FIG. 3C).

Due to the vibration of the tool 12, the hard tissue HT is cut from the side of an adhering surface of the soft tissue ST and the hard tissue HT, and the hard tissue HT is peeled from the soft tissue ST. As a result, the hard tissue HT can be removed in a shorter time period and more stably than when the hard tissue HT is cut until it becomes thin toward the soft tissue ST.

In the present embodiment, the hard tissue HT is a bone, an ossified cell, or the like of a predetermined portion of a human body, and the soft tissue ST is a nerve, a blood vessel, or the like adjacent to the hard tissue HT. Accordingly, the safety of the soft tissue ST needs to be ensured in the surgery.

In the present embodiment, the control device 20 controls the vibration of the tool 12 by the vibration device 21 to burst oscillation in which vibration and stop are repeated. As a result, the tool 12 can be more prevented from generating heat so that the safety of the soft tissue ST can be more ensured than when the tool 12 is always vibrated or a tool which is rotated.

The tool 12 is cooled with cooling water. Thus, the tool 12 can be further prevented from generating heat.

Further, the groove 12c is formed on the second surface 12b, which contacts the soft tissue ST, of the tool 12, and cooling water is also sent to the groove 12c. As a result, a cooling effect with cooling water can be enhanced.

Although the preferred embodiment of the present invention has been described above, the present invention is not limited to the embodiment, but is appropriately changeable without departing from the spirit of the present embodiment.

Although the tool 12 is cooled with cooling water in the above-described embodiment, for example, the cooling is not limited to the cooling with cooling water as long as the tool 12 can be cooled. For example, cooling gas may be sprayed onto the tool 12.

The groove 12c of the tool 12 may be extended toward a rear end portion of the tool 12 so that cooling water is directly poured into the groove 12c.

Although the first surface 12a of the tool 12 is formed in a circular arc shape and a concave shape in the above-described embodiment, the first surface 12a may be formed in a circular arc shape and a planar shape.

Further, although the groove 12c extending toward the distal end side from the proximal end (rear end) side of the tool 12 is formed on the second surface 12b of the tool 12 in the above-described embodiment, the shape of the groove 12c is appropriately changeable. For example, a spiral groove may be formed. Further, a plurality of grooves may be formed.

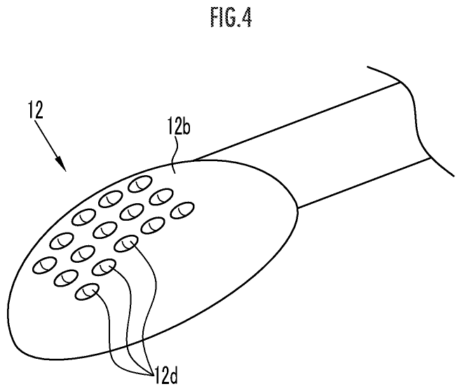

A plurality of recesses 12d as illustrated in FIG. 4 may be formed instead of or in addition to the groove 12c. In this case, a cooling effect can also be enhanced.

Further, a shape of the flow path 25 is appropriately changeable. For example, the flow path 25 may be formed in such a spiral shape as to surround the holding member 11. A flow path for causing cooling water to flow may be formed in the tool 12.

### REFERENCE SIGNS LIST

2 . . . vibration type removal apparatus, 10 . . . housing, 11 . . . holding member, 12 . . . tool, 20 . . . control device, 21 . . . vibration device, 22 . . . cooling pump, HT . . . hard tissue, ST . . . soft tissue

1. A vibration type removal apparatus that peels and removes a hard tissue from a soft tissue, the vibration type removal apparatus comprising:
a housing;
a vibration device arranged in the housing;
a tool provided in a distal end portion of the vibration device, protruding from the housing, and insertable between the hard tissue and the soft tissue;
a control device that controls driving of the vibration device to vibrate the tool in an axial direction of the vibration device; and
a cooling device that cools the tool,
wherein the control device controls the vibration of the tool by the vibration device to burst oscillation in which stop and vibration are repeated.

2. The vibration type removal apparatus according to claim 1, wherein
a first surface, which opposes the soft tissue, of the tool has a plurality of recesses or a groove extending toward a distal end side from a proximal end side of the tool formed thereon.

3. The vibration type removal apparatus according to claim 1, wherein
a second surface, which opposes the hard tissue, of the tool is formed in a circular arc shape, and also a planar shape or a concave shape.

4. The vibration type removal apparatus according to claim 1, wherein
the cooling device is composed of a cooling liquid supply device that supplies a cooling liquid, and
the housing is provided with a flow path that causes a cooling liquid supplied from the cooling liquid supply device to flow toward a distal end side of the tool.
